(19) United States
(12) Patent Application Publication
(10) Pub. No.: US 2026/0315124 A1
(43) Pub. Date: Oct. 15, 2026

(54) PEEK HA FUSION NAIL IMPLANT

(71) Applicant: Thomas Zink, San Antonio, TX (US)

(72) Inventor: Thomas Zink, San Antonio, TX (US)

(21) Appl. No.: 19/426,495

(22) Filed: Dec. 19, 2025

Related U.S. Application Data
(60) Provisional application No. 63/788,887, filed on Apr. 15, 2025.

(51) Int. Cl. A61B 17/84; A61B 17/00; A61B 17/56; A61B 17/88; A61B 17/92; A61B 50/30; A61B 90/00

(52) U.S. Cl. CPC A61B 17/846; A61B 17/8863; A61B 17/8872; A61B 17/8897; A61B 17/92; A61B 50/30; A61B 90/08; A61B 90/39; A61B 2017/00964; A61B 2017/564; A61B 2017/922; A61B 2090/0807; A61B 2090/3966

(57) ABSTRACT

A fusion nail implant is disclosed, which is used to connect and promote fusion in any surgical procedure where fusion between bones or bone fragments is desired. Any suitable number of fusion nail implants can be utilized to fixate the bones or bone fragments. Typically, the fusion nail implant comprises a body component configured in an elongated, cylindrical shape and manufactured of fusion-grade PEEK. The body component is cannulated for insertion of a k-wire and includes a plurality of ribs in a spaced apart manner for optimal fixation. Typically, a pair of opposing tantalum markers are positioned at an end for locating the implant under fluoroscopy.

### CROSS-REFERENCE TO RELATED APPLICATION

The present application claims priority to, and the benefit of, U.S. Provisional Application No. 63/788,887, which was filed on Apr. 15, 2025 and is incorporated herein by reference in its entirety.

### FIELD OF THE INVENTION

The present invention relates generally to a medical device for use in the field of podiatry. More particularly, the present invention relates to a PEEK HA fusion nail implant which is used to connect and promote fusion in any surgical procedure where fusion between bones or bone fragments is desired. Specifically, the implant is an osteochondral fixation device and is used as a screw or pin would be used to fixate the bones together. Accordingly, the present disclosure makes specific reference thereto. Nonetheless, it is to be appreciated that aspects of the present invention are also equally applicable to other like applications, devices and methods of manufacture.

### BACKGROUND

Fusion nails have long been used for internal fixation of an extremity and have become the preferred implant treatment in many bone fracture cases. Generally, the fusion nail is an osteochondral fixation device and is used as a screw or pin would be used to fixate the bones together. Such a device comprises an extended hollow shaft having a predetermined cross-section and provided with transverse ribs cut into the circumference, along its length to mitigate movement once placed. The fusion nail is inserted into any bone and impacted into place.

Because bone sizes vary, prior art fusion nails are produced in a variety of lengths and diameters. In order to limit the number of sizes which must be carried in inventory, the length of the fusion nail is trimmable per surgeon need. Further, the fusion nail implant is trimmable in situ as well as on the back table before it's implanted.

Previously, internal fixation is performed via screws and k-wires which are all typically made from metal, such as titanium or stainless steel. However, use of metal implants obscures X-rays and/or CT imaging. Further, these metal implants are hard to revise and difficult to cut through, if needed. Additionally, many patients may have allergies to such metal implants or may develop a metal allergy due to prolonged exposure to metals, such as through implantation of medical devices/implants. Accordingly, the use of PEEK (polyether-ether-ketone), which is radiolucent, in medical implants allows for X-rays and/or CT imaging to pass through without significant absorption, making it suitable for medical imaging. However, the use of (polyether-ether-ketone) PEEK in such implants does not allow for osteo-conduction, which is the growth of new bone along a surface of an implant or other structure. Therefore, fusion-grade PEEK has been formulated to allow for such osteo-conduction, while also maintaining its radiolucency.

Accordingly, it would be desirable to provide a fusion nail implant that is used to connect and promote fusion in any surgical procedure where fusion between bones or bone fragments is desired. Further, it would be desirable to provide a fusion nail implant that is trimmable to produce a variety of lengths, as needed and manufactured of fusion-grade PEEK.

The subject matter disclosed and claimed herein, in one embodiment thereof, comprises a fusion nail implant. The fusion nail implant is designed as an osteochondral fixation device which is used to connect and promote fusion in any surgical procedure where fusion between bones or bone fragments is desired. The fusion nail implant comprises a body component configured in an elongated, cylindrical shape and manufactured of fusion-grade PEEK. The body component comprises a proximal end, a distal end, and a curved sidewall connecting the two ends. The body component is cannulated for insertion of a k-wire. The curved sidewall includes a plurality of ribs in a spaced apart manner for optimal fixation. The plurality of ribs are cut into the circumference, along the body component's length. The distal end comprises a pair of opposing tantalum markers for locating the implant under fluoroscopy. Further, the distal end comprises a tapered or chamfered tip for easy insertion of the implant into bone.

In use, a k-wire is inserted across the fracture/osteotomy site until the distal trocar tip of the k-wire penetrates the desired depth. The fusion nail implant length is determined via a depth gauge on the k-wire. A canulated drill is slid over the k-wire and drilled to the desired depth. After the appropriate fusion nail implant diameter has been selected, the fusion nail implant is placed in a nail cutter and trimmed to a desired length. Then, the trimmed fusion nail implant is inserted over the k-wire and impacted into the bone until the desired depth is achieved. Finally, the k-wire is removed. The fusion nail implant can be removed, if needed, via an extraction drill and slap hammer to gradually persuade the implant out.

In this manner, the fusion nail implant of the present invention accomplishes all of the forgoing objectives and provides users with an implant that is manufactured of fusion-grade PEEK so it is inert and does not resorb into the surrounding bone. The fusion nail implant is a cylindrical nail that is cannulated to accept a k-wire placement, with the addition of tantalum markers positioned at the distal end for locating the implant under fluoroscopy.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some concepts in a simplified form as a prelude to the more detailed description that is presented later.

The subject matter disclosed and claimed herein, in one embodiment thereof, comprises a fusion nail implant. The fusion nail implant is designed as an osteochondral fixation device which is used to connect and promote fusion in any surgical procedure where fusion between bones or bone fragments is desired. The fusion nail implant comprises a body component configured in an elongated, cylindrical shape and manufactured of fusion-grade PEEK. The body component comprises a proximal end, a distal end, and a curved sidewall connecting the two ends. The body component is cannulated for insertion of a k-wire. The curved sidewall includes a plurality of ribs in a spaced apart manner for optimal fixation. The plurality of ribs are cut into the circumference, along the body component's length. The distal end comprises a pair of opposing tantalum markers for locating the implant under fluoroscopy. Further, the distal end comprises a tapered or chamfered tip for easy insertion of the implant into bone.

In one embodiment, the fusion nail implant comprises a body component configured in an elongated, cylindrical shape. The body component comprises a proximal end and a distal end that share a curved cylindrical sidewall extending therebetween. The body component is cannulated for accepting a k-wire.

In one embodiment, the implant is manufactured of fusion-grade PEEK. Any suitable fusion-grade PEEK can be utilized as is known in the art, but typically the fusion-grade PEEK is PEEK HA (hydroxyapatite) or PEEK composited with calcium-phosphate.

In one embodiment, the fusion nail implant may be implemented with a range of diameters that facilitate using the implant to fixate bones or bone fragments in various bone fractures or osteotomies in the human body, such as by way of non-limiting example, an akin osteotomy, a chevron osteotomy, a lesser metatarsal fracture, a calcaneal fracture, a scarf osteotomy, a supramalleolar fracture, as well as any other extremity fractures or osteotomies of the hand or foot as is known in the art. It is contemplated, however, that the overall size of the implant is to be selected according to the particular bones or bone fragments to be fixated.

In one embodiment, the implant possesses a length along the axis of the implant and a diameter centered on the axis. The length extends from the proximal end to the distal end. In one embodiment, the length is substantially 30-120 millimeters (mm). It is contemplated, however, that the length may be varied according to the bone or bone fragment to be fixated, and thus the implant may be implemented with a wide variety of lengths, without limitation. Further, typically the length of the body component can be trimmed with a cutter, as needed to create the exact size desired.

In one embodiment, the diameter of the proximal end is substantially the same as the diameter of the distal end, and thus the cylindrical sidewall comprises a straight cylindrical shape, without limitation. In one embodiment, the diameter is substantially 2.5-8 millimeters (mm). It is contemplated, however, that the diameter may be varied according to the bone or bone fragment to be fixated, and thus the implant may be implemented with a wide variety of diameters, without limitation. In such embodiments, a practitioner may select the implant based on a size of the bone or bone fragment to be fixated.

In one embodiment, when the fusion nail implant is implanted into the bone or bone fragment, the proximal end of the implant is disposed flush or slightly below the surrounding cortical bone. For fusion nail implants, the proximal end has a flat curvature as the implant generally is disposed flush with the surrounding cortical bone.

In one embodiment, the cylindrical sidewall comprises a plurality of ribs in a spaced apart manner for optimal fixation. The plurality of ribs are cut into the circumference, along the body component's length. The ribs are cut in, such that the surrounding bone enters between the ribs when the implant is inserted. Typically, the ribs are cut in approximately ¼-½ mm radially. Any suitable number of ribs can be utilized as is known in the art. Further, the ribs are configured in a spaced apart manner. Typically, the ribs are spaced apart, such that they have a pitch of approximately 2-3 millimeters to promote boney apposition. Upon insertion, the fusion nail implant is positioned axially or perpendicularly over the bone or bone fragment and persuaded into place via a mallet and insertion tamp. This makes it possible to manufacture the implant out of a radiolucent polymer, such as PEEK, which performs well in compression, especially when being persuaded into place via the mallet. The use of PEEK does not do well with a torsional load, as such a user would not want to twist or torque the implant into the hole, as in the prior screw technology. The PEEK material is radiolucent/radiotransparent, which will allow for better post-surgery monitoring using x-ray imaging. Also, PEEK is more workable by drilling or cutting with various surgical instruments.

In one embodiment, the distal end of the body component comprises a pair of opposing tantalum markers for locating the implant under fluoroscopy. The tantalum markers can be any suitable shape and size as is known in the art, as long as the tantalum markers are shaped and sized to fit on the distal end of the body component. Further, the tantalum markers can be positioned in any suitable position on the body component. Typically, the tantalum markers are positioned on the distal end that enters the bone/bone fragment, such that if the user trims the proximal end, they don't cut off the tantalum markers. Additionally, any suitable number of tantalum markers can be utilized as is known in the art, depending on the needs and/or wants of a user.

In one embodiment, the distal end of the body component comprises a tapered or chamfered tip for easy insertion of the implant into bone. The tapered tip can be any desired angle or length as is known in the art. More specifically, the angle of the tapered tip is approximately between 30-120 degrees and the length of the tapered tip is approximately between 1-2 mm in length.

In one embodiment, the fusion nail implant system for fixating bones and bone fragments according to the present disclosure includes one or more fusion nail implants, a k-wire, a depth gauge, a cannulated drill, a nail cutter, a mallet, and insertion tamp. As will be appreciated, the fusion nail implant system comprises instruments necessary for fixating bones and bone fragments by way of surgery. The sizes of the instruments comprising the implant system will depend upon the size of the particular implant to be implanted into the patient. It is envisioned, therefore, that a surgeon may select the implant and a correspondingly sized instrument based on the location and size of the bone/bone fragment to be fixated. The instruments comprising the fusion nail implant system are not to be limited to the specific instruments, or the sizes and shapes of the instruments disclosed.

In operation, first, a k-wire is inserted across the fracture/osteotomy site until the distal trocar tip of the k-wire penetrates the far cortex or until the desired depth is achieved. Next, the correct placement is verified using fluoroscopy. Then, a depth gauge is slid onto the k-wire until it reaches the near cortex to determine the appropriate nail length. Then, the cannulated drill is slid over the k-wire and used to drill to a desired depth. The fusion nail implant is then cut to a desired size and inserted over the k-wire and impacted into the bone. Specifically, the fusion nail implant is installed using the insertion tamp and a mallet until a desired depth is achieved. Finally, the insertion tamp and k-wire are removed.

In one embodiment, the k-wire comprises an elongated shaft having a distal pointed tip and a proximal blunt end. The k-wire is available in multiple diameters and lengths and is configured to be inserted into confined spaces within the bone or bone fragments and serves to direct a subsequent insertion of the cannulated drill and the cannulated fusion nail implant to the implant location within the bone/bone fragment. It will be appreciated that the distal pointed tip facilitates advancing the k-wire through obstructive tissues and structures, and the proximal blunt end facilitates manipulating the k-wire by hand, or by way of an appropriate tool.

In one embodiment, the cannulated drill comprises a rigid elongate shaft having a distal cutting end and a proximal shank. The distal cutting end comprises a cutting edge suitable for rotatably cutting bone. The proximal shank is configured to be grasped by a chuck of a surgical drill, or other equivalent rotary tool. Further, in some embodiments the cannulated drill comprises a central, lengthwise hole whereby the drill may be mounted onto the k-wire so as to direct the distal cutting end to the location on the bone.

In one embodiment, the nail cutter comprises a graduated measurement guide which is cannulated. The fusion nail implant is then inserted into the cannulated opening and retained via a retaining detent that engages the body component. Further, the outside length of the measurement guide includes millimeter markings ranging from 0-110 mm to aid a user in accurately cutting the fusion nail implant to the desired length. Further, a saw blade guide is secured to the distal end of the graduated measurement guide. The saw blade guide includes a slot or opening for accepting a sagittal saw to cut the fusion nail implant to the desired length.

In one embodiment, the mallet is a flat-ended hammer, which is used with the insertion tamp to drive the fusion nail implant into the bone. Once inserted, the implant is flush with or sub-flush to the surrounding cortical bone.

In general, it is contemplated that the fusion nail implant system is to be suitably sterilized for surgeries and packaged into sterilized containers.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIGS. 1A-1C illustrate a perspective view of the fusion nail implant of the present invention in accordance with the disclosed architecture;

FIG. 2 illustrates a perspective view of the procedure for inserting the fusion nail implant of the present invention showing the k-wire in accordance with the disclosed architecture;

FIG. 3 illustrates a perspective view of the procedure for inserting the fusion nail implant of the present invention showing the cannulated drill in accordance with the disclosed architecture;

FIG. 4 illustrates a perspective view of the procedure for inserting the fusion nail implant of the present invention showing the cannulated drill in accordance with the disclosed architecture

FIGS. 5A-5E illustrate a perspective view of the procedure for inserting the fusion nail implant of the present invention showing the nail cutter in accordance with the disclosed architecture;

FIG. 6 illustrates a perspective view of the procedure for inserting the fusion nail implant of the present invention showing the mallet and insertion tamp in accordance with the disclosed architecture;

FIG. 7 illustrates a perspective view of the procedure for inserting the fusion nail implant of the present invention showing the mallet and insertion tamp in accordance with the disclosed architecture;

FIG. 8 illustrates a perspective view of the procedure for inserting the fusion nail implant of the present invention showing the extraction drill in accordance with the disclosed architecture; and

FIGS. 9A-9B illustrate a perspective view of the procedure for inserting the fusion nail implant of the present invention showing the slap hammer in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

In this application, the words proximal, distal, anterior or plantar, posterior or dorsal, medial and lateral are defined by their standard usage for indicating a particular part or portion of a bone, or directional terms of reference, according to the relative disposition of the natural bone. For example, “proximal” means the portion of a bone nearest the torso, while “distal” indicates the portion of the bone farthest from the torso. As an example of directional usage of the terms, “anterior” refers to a direction towards the front side of the body, “posterior” refers to a direction towards the back side of the body, “medial” refers to a direction towards the midline of the body and “lateral” refers to a direction towards the sides or away from the midline of the body. Further, specifically in regard to the foot, the term “dorsal” refers to the top of the foot and the term “plantar” refers the bottom of the foot.

Similarly, positions or directions may be used herein with reference to anatomical structures or surfaces. For example, as the current devices, instrumentation and methods are described herein with reference to use with the bones of the foot, the bones of the foot, ankle and lower leg may be used to describe the surfaces, positions, directions or orientations of the devices, instrumentation and methods. Further, the devices, instrumentation and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the devices, instrumentation and methods, and the aspects, components, features and the like thereof, described herein with respect to the right foot may be mirrored so that they likewise function with the left foot. Further, the devices, instrumentation and methods, and the aspects, components, features and the like thereof, disclosed herein are described with respect to the foot for brevity purposes, but it should be understood that the devices, instrumentation, and methods may be used with other bones of the body having similar structures, for example the upper extremity, and more specifically, with the bones of the wrist, hand, and arm.

As noted above, there is a long felt need in the art for devices, instrumentation, and methods of inserting the fusion nail implant. Specifically, devices, instrumentation and methods used to fixate bones or bone fragments together.

The present invention, in one exemplary embodiment, is a novel fusion nail implant. The implant fixates bones or bone fragments together to allow for fusion of the bones. The fusion nail implant comprises an elongated, cylindrical structure which is cannulated and includes a plurality of ribs along its length. The instrumentation includes at least a k-wire, a cannulated drill, a nail cutter, and an inserter, among other instruments.

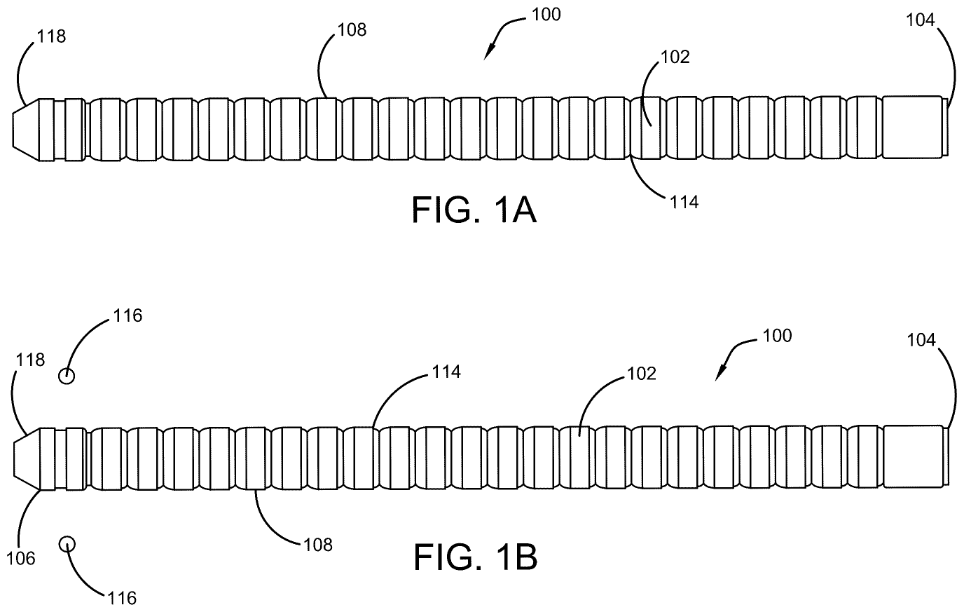

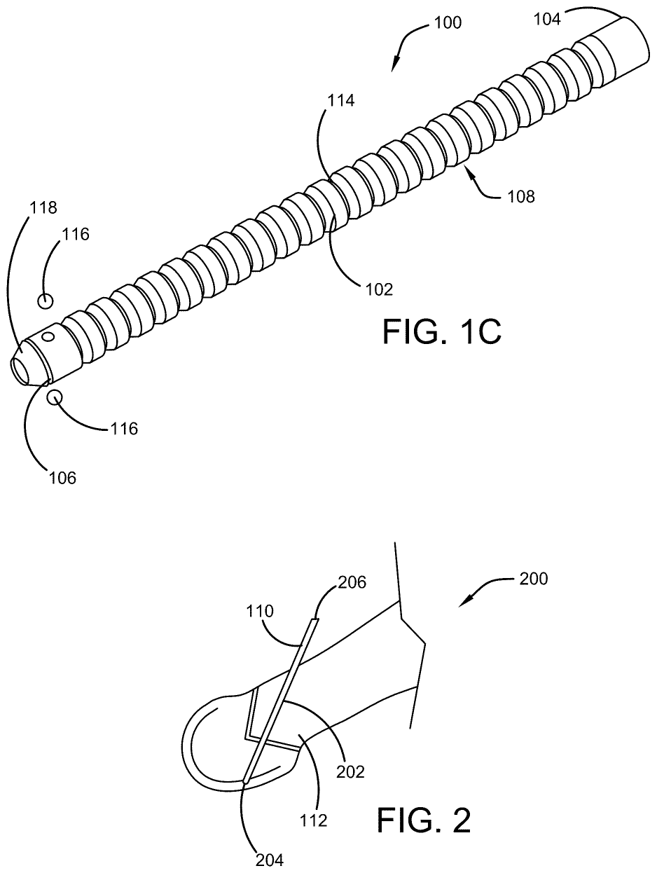

Referring initially to the drawings, FIGS. 1A-C illustrate multiple views of the fusion nail implant 100 of the present invention. In the present embodiment, the fusion nail implant 100 is an improved fusion nail implant 100 that fixates bones or bone fragments together. The implant 100 is especially designed to allow any doctor, surgeon, etc., or any other suitable user as is known in the art, to fixate the bones together, providing for bone fusion. More specifically, the fusion nail implant system 500 comprises the fusion nail implant 100 and instrumentation for inserting the fusion nail implant 100.

The fusion nail implant 100 comprises a body component 102 configured in an elongated, cylindrical shape. The body component 102 comprises a proximal end 104 and a distal end 106 that share a curved cylindrical sidewall 108 extending therebetween. The body component 102 is cannulated for accepting a k-wire 110. During use, the cannulated body component 102 is inserted onto the k-wire 110 then impacted into the bone 112.

The implant 100 is manufactured of fusion-grade PEEK. Any suitable fusion-grade PEEK can be utilized as is known in the art, but typically the fusion-grade PEEK is PEEK HA (hydroxyapatite) or PEEK composited with calcium phosphate.

Further, the fusion nail implant 100 may be implemented with a range of diameters that facilitate using the implant 100 to fixate bones 112 or bone fragments in various bone fractures or osteotomies in the human body, such as by way of non-limiting example, an akin osteotomy, a chevron osteotomy, a lesser metatarsal fracture, a calcaneal fracture, a scarf osteotomy, a supramalleolar fracture, as well as any other extremity fractures or osteotomies of the hand or foot as is known in the art. It is contemplated, however, that the overall size of the implant 100 is to be selected according to the particular bones 112 or bone fragments to be fixated. Further, any suitable number of fusion nail implants 100 can be utilized to fixate bones 112 or bone fragments in the various bone fractures or osteotomies in the human body, depending on the size of the bone 112 or bone fragment to be fixated and/or the surgeon or user's wants and/or needs.

Additionally, the implant 100 possesses a length along the axis of the implant 100 and a diameter centered on the axis. The length extends from the proximal end 104 to the distal end 106. In one embodiment, the length is substantially 30-120 millimeters (mm). It is contemplated, however, that the length may be varied according to the bone 112 or bone fragment to be fixated, and thus the implant 100 may be implemented with a wide variety of lengths, without limitation. Further, typically the length of the body component 102 can be trimmed with a cutter 500, as needed to create the exact size desired. Specifically, after the appropriate nail implant 100 diameter has been selected, the nail implant 100 can be placed in a nail cutter 500 and trimmed to a desired length using a standard sagittal saw 502 during surgery. Thus, the fusion nail implant 100 is then cut to a desired size and inserted over the k-wire 110 and impacted into the bone 112. Accordingly, the nail implant 100 allows a user to customize the length of the nail implant 100 depending on the size of the bone 112 or bone fragments to fixate and can be trimmed before or after insertion within the bone 112 (i.e., in situ trimming).

Further, the diameter of the proximal end 104 is substantially the same as the diameter of the distal end 106, and thus the cylindrical sidewall 108 comprises a straight cylindrical shape, without limitation. The cylindrical nature of the body component 102 provides for more surface area contact between the nail implant 100 and the surrounding bone, which provides additional osseointegration for the implant 100. In one embodiment, the diameter is substantially 2.5-8 millimeters (mm). It is contemplated, however, that the diameter may be varied according to the bone 112 or bone fragment to be fixated, and thus the implant 100 may be implemented with a wide variety of diameters, without limitation. In such embodiments, a practitioner may select the implant 100 based on a size of the bone 112 or bone fragment to be fixated.

Generally, the implant 100 is available in multiple sizes, such as an implant 100 with a 3.0 mm diameter has a length of approximately 50 mm; an implant 100 with a 4.0 mm diameter has a length of approximately 70 mm; an implant 100 with a 5.0 mm diameter has a length of approximately 100 mm; and an implant 100 with a 6.0 mm diameter has a length of approximately 100 mm.

Furthermore, when the fusion nail implant 100 is implanted into the bone 112 or bone fragment, the proximal end 104 of the implant 100 is disposed flush or slightly below the surrounding cortical bone. For fusion nail implants 100, the proximal end 104 has a flat curvature as the implant 100 generally is disposed flush with the surrounding cortical bone.

Additionally, the cylindrical sidewall 108 comprises a plurality of ribs 114 in a spaced apart manner for optimal fixation. The plurality of ribs 114 are cut into the circumference, along the body component's length. The ribs 114 are cut in, such that the surrounding bone 112 enters between the ribs 114 when the implant 100 is inserted. Typically, the ribs 114 are cut in approximately ¼-½ mm radially. Any suitable number of ribs 114 can be utilized as is known in the art. Further, the ribs 114 are configured in a spaced apart manner. Typically, the ribs 114 are spaced apart, such that they have a pitch of approximately 2-3 millimeters to promote boney apposition. Upon insertion, the fusion nail implant 100 is positioned axially or perpendicularly over the bone 112 or bone fragment and persuaded into place via a mallet 600 and insertion tamp 602. This makes it possible to manufacture the implant 100 out of a radiolucent polymer, such as PEEK, which performs well in compression, especially when being persuaded into place via the mallet 600. The use of PEEK does not do well with a torsional load, as such a user would not want to twist or torque the implant 100 into the hole, as in the prior screw technology. Further, impaction is better for fixation of bones (i.e., fusion), whereas torquing or twisting an implant into position is better for compression. The PEEK material is radiolucent/radiotransparent, which will allow for better post-surgery monitoring using x-ray imaging. Also, PEEK is more workable by drilling or cutting with various surgical instruments.

Further, the distal end 106 of the body component 102 comprises a pair of opposing tantalum markers 116 for locating the implant 100 under fluoroscopy. The tantalum markers 116 can be any suitable shape and size as is known in the art, as long as the tantalum markers 116 are shaped and sized to fit on the distal end 106 of the body component 102. Further, the tantalum markers 116 can be positioned in any suitable position on the body component 102. Typically, the tantalum markers 116 are positioned on the distal end 106 that enters the bone 112/bone fragment, such that if the user trims the proximal end 104, they don't cut off the tantalum markers 116. Additionally, any suitable number of tantalum markers 116 can be utilized as is known in the art, depending on the needs and/or wants of a user.

Additionally, the distal end 106 of the body component 102 comprises a tapered or chamfered tip 118 for easy insertion of the implant 100 into bone 112. The tapered tip 118 can be any desired angle or length as is known in the art. More specifically, the angle of the tapered tip 118 is approximately between 30-120 degrees and the length of the tapered tip 118 is approximately between 1-2 mm in length.

As shown in FIGS. 2-9, the fusion nail implant system 200 for fixating bones 112 and bone fragments according to the present disclosure includes one or more fusion nail implants 100, a k-wire 110, a depth gauge 300, a cannulated drill 400, a nail cutter 500, a mallet 600, and insertion tamp 602. As will be appreciated, the fusion nail implant system 200 comprises instruments 110, 300, 400, 500, 600, 602 necessary for fixating bones 112 and bone fragments by way of surgery. The sizes of the instruments 110, 300, 400, 500, 600, 602 comprising the implant system 200 will depend upon the size of the particular implant 100 to be implanted into the patient. It is envisioned, therefore, that a surgeon may select the implant 100 and a correspondingly sized instrument based on the location and size of the bone 112/bone fragment to be fixated. The instruments 110, 300, 400, 500, 600, 602 comprising the fusion nail implant system 200 are not to be limited to the specific instruments, or the sizes and shapes of the instruments disclosed.

A surgical method for fixating bones/bone fragments together, will now be described. The method utilizes some of the devices, instruments, features, aspects, components and the like described above, and therefore reference will be made to the above-described embodiments, such as the illustrated embodiments presented in the figures and discussed above. However, such references are made for exemplary purposes only and are not intended to limit the surgical method beyond the specifically recited steps. Further, the surgical method may be discussed under the umbrella of particular bones, but such an application is not intended to be limiting and the method described herein may be used or conducted with bone or other tissue not specifically discussed herein without departing from the spirit and scope of the surgical method.

In operation, first, a k-wire 110 is inserted across the fracture/osteotomy site until the distal trocar tip of the k-wire 110 penetrates the far cortex or until the desired depth is achieved. Next, the correct placement is verified using fluoroscopy. Then, a depth gauge 300 is slid onto the k-wire 110 and slid forward until it reaches the near cortex to determine the appropriate nail implant 100 length. Then, the appropriate cannulated drill 400 is slid over the k-wire 110 based on the nail implant 100 diameter to be used. The cannulated drill 400 is used to drill to a desired depth or to the far cortex surface to facilitate bi-cortical fixation. A drill guide can be used to verify measurement depth of the drill 400. After the appropriate nail implant 100 diameter has been selected, the nail implant 100 can be placed in a nail cutter 500 and trimmed to a desired length using a standard sagittal saw 502. Specifically, the fusion nail implant 100 is then cut to a desired size and inserted over the k-wire 110 and impacted into the bone 112. Further, the fusion nail implant 100 is inserted over the k-wire 110 with a corresponding inserter or insertion tamp 602. Specifically, the insertion tamp 602 is positioned on the proximal end 104 of the implant 100 and impacted into the bone 112 using the mallet 600 until a desired depth is achieved. Finally, the insertion tamp 602 and k-wire 110 are removed.

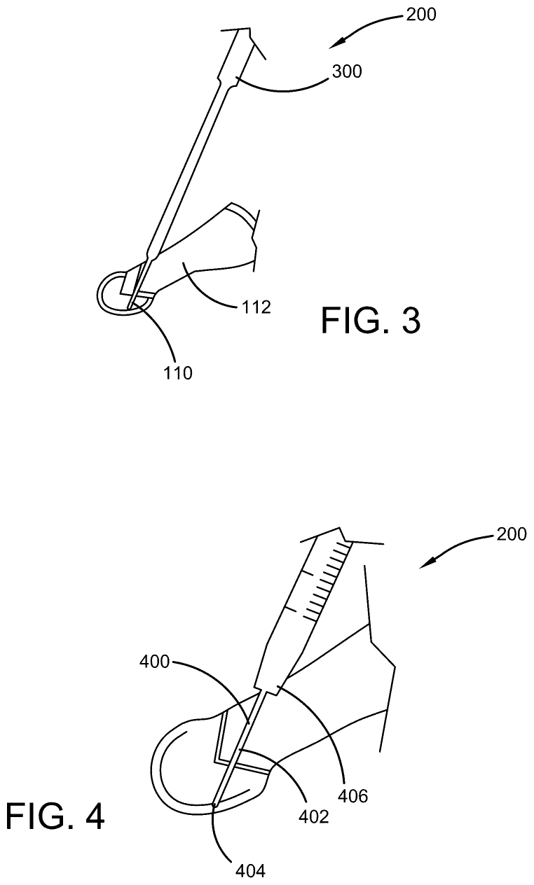

Generally, as shown in FIGS. 2-3, the k-wire 110 comprises an elongated shaft 202 having a distal pointed tip 204 and a proximal blunt end 206. The k-wire 110 is available in multiple diameters and lengths and is configured to be inserted into confined spaces within the bone 112 or bone fragments and serves to direct a subsequent insertion of the cannulated drill 400 and the cannulated fusion nail implant 100 to the implant location within the bone 112/bone fragment. It will be appreciated that the distal pointed tip 204 facilitates advancing the k-wire 110 through obstructive tissues and structures, and the proximal blunt end 206 facilitates manipulating the k-wire 110 by hand, or by way of an appropriate tool.

As shown in FIG. 4, the cannulated drill 400 comprises a rigid elongate shaft 402 having a distal cutting end 404 and a proximal shank 406. The distal cutting end 404 comprises a cutting edge suitable for rotatably cutting bone. The proximal shank 406 is configured to be grasped by a chuck of a surgical drill, or other equivalent rotary tool. Further, in some embodiments the cannulated drill 400 comprises a central, lengthwise hole whereby the drill 400 may be mounted onto the k-wire 110 so as to direct the distal cutting end 404 to the location on the bone 112.

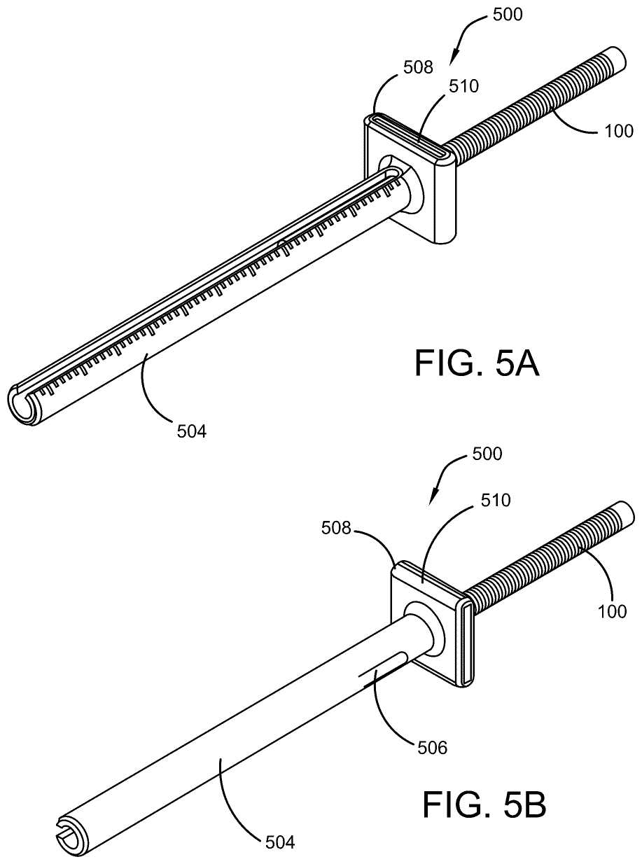

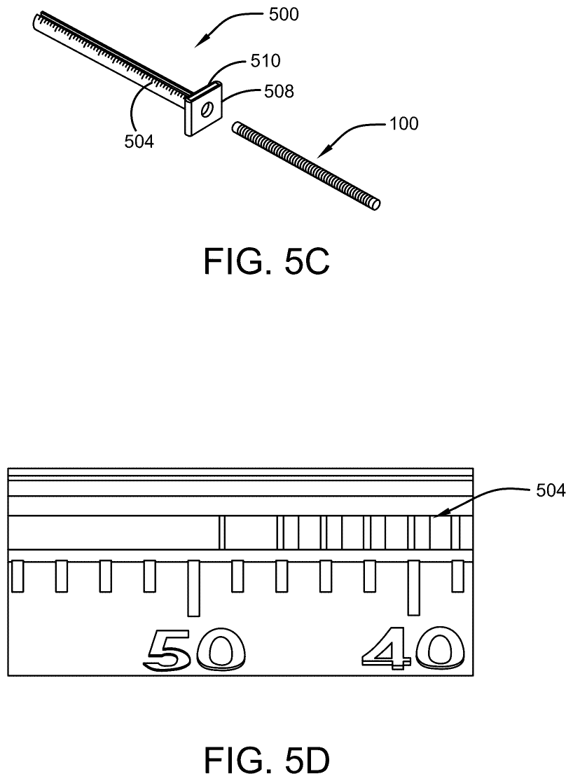

As shown in FIGS. 5A-E, the nail cutter 500 comprises a graduated measurement guide 504 which is cannulated. The fusion nail implant 100 is then inserted into the cannulated opening and retained via a retaining detent 506 that engages the body component 102. Further, the outside length of the measurement guide 504 includes millimeter markings ranging from 0-110 mm to aid a user in accurately cutting the fusion nail implant 100 to the desired length. Further, a saw blade guide 508 is secured to the distal end of the graduated measurement guide 504. The saw blade guide 508 includes a slot or opening 510 for accepting a sagittal saw 502 to cut the fusion nail implant 100 to the desired length.

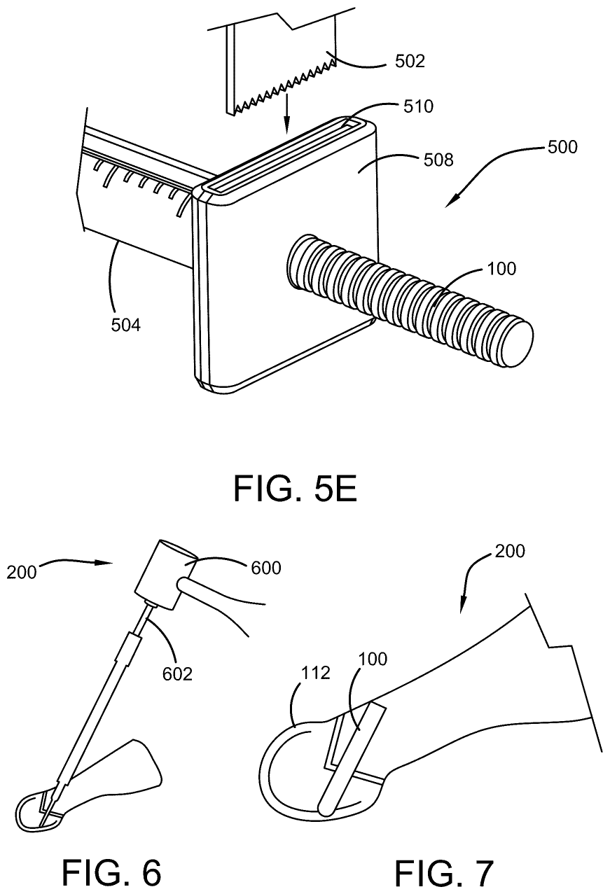

As shown in FIGS. 6-7, the mallet 600 is a flat-ended hammer, which is used with the insertion tamp 602 to drive the fusion nail implant 100 into the bone 112. Once inserted, the implant 100 is flush with or sub-flush to the surrounding cortical bone.

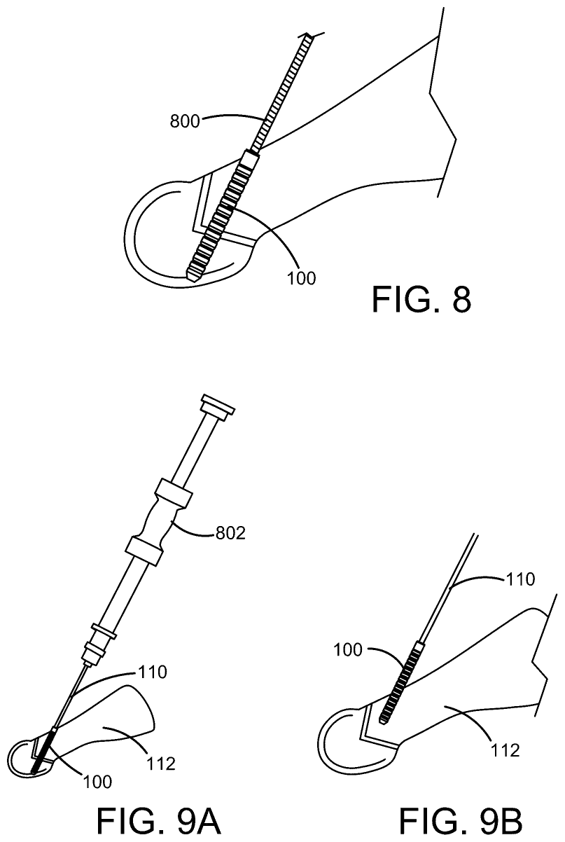

As shown in FIGS. 8 and 9A-B, for removal of the fusion nail implant 100 as needed, an extraction drill 800 is inserted into the cannulation of the nail implant 100. The extraction drill 800 can be either 2.0 mm for 3.0-4.5 mm implants or 2.5 mm for 5.0-7.0 mm implants. Further, the extraction drill 800 should engage the entire length of the implant 100. Once the extraction drill 800 is fully inserted, the extraction drill 800 is disconnected from the handle or power and a slap hammer 802 is attached to the AO connect (i.e., power). The slap hammer 802 is engaged to gradually persuade the implant 100 out.

In general, it is contemplated that the fusion nail implant system 200 is to be suitably sterilized for surgeries and packaged into sterilized containers.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “fusion nail implant”, “nail implant”, and “implant” are interchangeable and refer to the fusion nail implant 100 of the present invention.

Notwithstanding the forgoing, the fusion nail implant 100 of the present invention can be of any suitable size and configuration as is known in the art without affecting the overall concept of the invention, provided that it accomplishes the above stated objectives. One of ordinary skill in the art will appreciate that the fusion nail implant 100 as shown in FIGS. 1-9 is for illustrative purposes only, and that many other sizes and shapes of the fusion nail implant 100 are well within the scope of the present disclosure. Although the dimensions of the fusion nail implant 100 are important design parameters for user convenience, the fusion nail implant 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A fusion nail implant comprising:
a body component configured in an elongated cylindrical shape;
wherein the body component comprises a proximal end and a distal end with a curved cylindrical sidewall extending therebetween;
wherein the body component is cannulated for insertion of a k-wire; and
further wherein during use, the body component is inserted on the k-wire, then impacted into a patient's bone for fixation.

2. The fusion nail implant of claim 1, wherein the body component is manufactured of a fusion-grade PEEK, a PEEK HA (hydroxyapatite) or a PEEK composited with a calcium phosphate.

3. The fusion nail implant of claim 1, wherein the body component comprises a length of substantially 30-120 millimeters.

4. The fusion nail implant of claim 3, wherein the length may be varied according to the patient's bone to be fixated.

5. The fusion nail implant of claim 4, wherein the length of the body component is configured to be trimmed with a cutter as needed to create an exact size desired before or after insertion of the fusion nail implant into the patient's bone.

6. The fusion nail implant of claim 1, wherein a diameter of the proximal end is substantially the same as a diameter of the distal end.

7. The fusion nail implant of claim 1, wherein the body component comprises a diameter of between 2.5-8 millimeters.

8. The fusion nail implant of claim 1, wherein when the body component is implanted into the patient's bone, the proximal end has a flat curvature that is disposed flush with, or slightly below, a surrounding portion of the patient's bone.

9. The fusion nail implant of claim 1, wherein the curved cylindrical sidewall comprises a plurality of ribs in a spaced apart manner.

10. The fusion nail implant of claim 9, wherein the plurality of ribs are cut into a circumference, along the body component's length, such that a surrounding portion of the patient's bone enters between the plurality of ribs when the body component is inserted.

11. The fusion nail implant of claim 10, wherein the plurality of ribs are cut into the curved cylindrical sidewall in approximately ¼-½ mm radially and have a pitch of approximately 2-3 millimeters.

12. The fusion nail implant of claim 1, wherein the distal end of the body component comprises a pair of opposing tantalum markers for locating the fusion nail implant under fluoroscopy.

13. The fusion nail implant of claim 1, wherein the distal end of the body component comprises a tapered tip for easy insertion of the fusion nail implant into the patient's bone.

14. The fusion nail implant of claim 13, wherein an angle of the tapered tip is approximately between 30-120 degrees, and a length of the tapered tip is approximately between 1-2 millimeters in length.

15. A fusion nail implant system that fixates a patient's bone or bone fragments together when in use, the fusion nail implant system comprising:
at least one fusion nail implant having a cannulated body component comprising a proximal end and a distal end with a curved cylindrical sidewall extending therebetween;
a k-wire for positioning the at least one fusion nail implant into the patient's bone or bone fragments;
a depth gauge to determine a desired length for the at least one fusion nail implant;
a cannulated drill for drilling to a desired depth in the patient's bone or bone fragments;
a nail cutter for cutting the at least one fusion nail implant to the desired length;
a mallet for impacting the at least one fusion nail implant into the patient's bone or bone fragments; and
an insertion tamp positioned on the proximal end of the body component to persuade the at least one fusion nail implant with the mallet into the patient's bone or bone fragments such that, once inserted, the proximal end is flush with or sub-flush to a surrounding portion of the patient's bone or bone fragments;
wherein the cannulated body component is manufactured of a fusion-grade PEEK, a PEEK HA (hydroxyapatite) or a PEEK composited with a calcium phosphate;
wherein the curved cylindrical sidewall of the cannulated body component comprises a plurality of ribs in a spaced apart manner; and
further wherein the distal end of the cannulated body component comprises a pair of opposing tantalum markers for locating the at least one fusion nail implant under fluoroscopy.

16. The fusion nail implant system of claim 15, wherein the fusion nail implant system is suitably sterilized for a surgical setting and packaged into a sterilized container.

17. A surgical method for fixating a patient's bone or bone fragment, the method comprising the following steps:
inserting a k-wire through the patient's bone or bone fragment to be fixated;
utilizing a cannulated drill to drill to a desired depth in the patient's bone or bone fragment;
cutting a fusion nail implant to a desired length via a nail cutter;
inserting the fusion nail implant over the k-wire;
installing the fusion nail implant using an insertion tamp and a mallet; and
removing the insertion tamp and the k-wire after the step of installing.

18. The method of claim 17, wherein after a k-wire is inserted, a depth gauge is slid onto the k-wire and slid forward until it reaches a near cortex to determine an appropriate length of the fusion nail implant.

19. The method of claim 18, wherein the insertion tamp is positioned on a proximal end of a body component of the fusion nail implant, which is then impacted into the patient's bone or bone fragment using the mallet until a desired depth is achieved, wherein once inserted into the patient's bone or bone fragment bone, the proximal end is flush with or sub-flush to a surrounding portion of the patient's bone or bone fragment.

20. The method of claim 19, wherein if removal of the fusion nail implant is needed, an extraction drill is inserted into a cannulated portion of the fusion nail implant and then a slap hammer is engaged to gradually persuade the fusion nail implant out of the patient's bone or bone fragment.
